(19) United States
(12) Patent Application Publication
(10) Pub. No.: US 2026/0166532 A1
(43) Pub. Date: Jun. 18, 2026

(54) ZEOLITE-BASED COMPOSITION, METHOD FOR PRODUCING ZEOLITE-BASED COMPOSITION, AND METHOD FOR PRODUCING LIGHT OLEFIN COMPOUND

(71) Applicant: Braskem America, Inc., Philadelphia, PA (US)

(72) Inventors: Samir Budhathoki, Philadelphia, PA (US); Brianna Collins, Philadelphia, PA (US); Ishant Khurana, Philadelphia, PA (US); Dan Xie, Philadelphia, PA (US); Graham Gregorich, Philadelphia, PA (US)

(73) Assignee: Braskem America, Inc., Philadelphia, PA (US)

(21) Appl. No.: 19/422,175

(22) Filed: Dec. 16, 2025

Related U.S. Application Data
(60) Provisional application No. 63/734,500, filed on Dec. 16, 2024.

(51) Int. Cl. B01J 29/06; B01J 29/70; B01J 35/61; C07C 5/333; C07C 11/02; C07C 11/12

(52) U.S. Cl. CPC B01J 29/061; B01J 29/7034; B01J 35/612; C07C 5/3335; C07C 11/02; C07C 11/12; C07C 2529/70

(57) ABSTRACT

The present disclosure relates to a catalyst composition comprising a metal catalyst chemically interacted with a zeolite comprising at least one zeolite framework selected from the group of clusters consisting of MTW framework cluster. The disclosure also relates to a catalyst composition comprising at least one alkaline-earth metal catalyst chemically interacted with a zeolite comprising at least one zeolite framework. The catalyst compositions described are useful for catalytic oxidative dehydrogenation of hydrocarbons.

### FIELD OF INVENTION

The present disclosure relates generally to chemical conversion processes, and more particularly to a catalyst system for converting light alkanes into light olefins using zeolites and/or alkaline-earth metal catalysts.

### BACKGROUND

Light alkanes are abundant hydrocarbon resources that are often underutilized in industrial processes. These compounds, including ethane, propane, and butane, are primarily obtained from natural gas and petroleum refining. Light alkanes have traditionally been used as fuel sources or as feedstocks for the production of other chemicals. However, their potential as precursors for more valuable products, such as light olefins, has gained significant attention in recent years. Light olefins are crucial building blocks in the petrochemical industry. These unsaturated hydrocarbons serve as key monomers for the production of various plastics, synthetic rubbers, and other polymeric materials. The demand for light olefins has been steadily increasing due to the growing global consumption of plastic products and the expansion of manufacturing industries.

The conversion of light alkanes into light olefins has been a longstanding challenge in the chemical industry. Traditional methods for this transformation have relied on energy-intensive processes such as steam cracking or catalytic cracking. These processes typically require high temperatures and pressures, resulting in significant energy consumption and associated greenhouse gas emissions. Additionally, the harsh reaction conditions often lead to the formation of unwanted byproducts, reducing the overall efficiency of the conversion process.

One of the major problems associated with conventional light alkane conversion methods is their environmental impact. The high energy requirements of these processes contribute to substantial carbon dioxide emissions, exacerbating concerns about climate change and environmental sustainability. Furthermore, the production of unwanted byproducts not only reduces the yield of desired olefins but also necessitates additional separation and purification steps, further increasing the overall environmental footprint of the process.

Another significant challenge in light alkane conversion is the economic viability of the process. The high operating costs associated with traditional methods, including energy consumption, catalyst replacement, and equipment maintenance, often result in reduced profit margins for manufacturers. This economic pressure has driven the search for more cost-effective and efficient conversion technologies that can maintain or improve product yields while minimizing operational expenses.

Alternative approaches to light alkane conversion have been explored in an attempt to address these challenges. Oxidative dehydrogenation (ODH) has emerged as a promising technique, offering the potential for lower energy requirements and improved selectivity compared to traditional cracking methods. However, the development of efficient and stable catalysts for ODH reactions remains an active area of investigation, as many existing catalysts suffer from rapid deactivation or insufficient catalytic activity under industrial conditions.

Therefore, there is a need to overcome the problems discussed above. A more sustainable and cost-effective method for converting light alkanes into light olefins is required to address the environmental and economic challenges associated with current processes. Such a method should aim to reduce energy consumption, minimize greenhouse gas emissions, improve catalytic activity towards desired olefin products, and enhance overall process efficiency. The development of innovative catalytic systems can achieve these goals remains a critical objective in the field of hydrocarbon conversion and utilization.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In a first aspect, the present disclosure provides a catalyst composition comprising a metal catalyst chemically interacted with a zeolite comprising at least one zeolite framework selected from the group of clusters consisting of MTW framework cluster, wherein the MTW framework cluster comprises zeolite frameworks having a Euclidean distance of 2.45 Ångström or less from the MTW framework.

In a second aspect, the present disclosure relates to a catalyst composition comprising at least one metal catalyst chemically interacted with a zeolite comprising at least one zeolite framework, wherein the at least one metal catalyst is selected from alkaline earth metal.

In another aspect, the present disclosure relates to a process for preparing the catalyst composition, which comprises adding a metal catalyst precursor to a zeolite comprising at least one zeolite framework selected from the MTW framework cluster to form a catalyst precursor mixture. The catalyst precursor mixture is then heated to a temperature of from 390° C. to 750° C. to form the catalyst composition

In another aspect, the present disclosure relates to a process for preparing the catalyst composition, which comprises adding at least one alkaline earth metal catalyst precursor to a zeolite comprising at least one zeolite framework to form a catalyst precursor mixture. The catalyst precursor mixture is then heated to a temperature of from 390° C. to 750° C. to form the catalyst composition

In another aspect, the present disclosure relates to a process for catalytic oxidative dehydrogenation of hydrocarbons, which involves contacting a hydrocarbon-containing feedstock with a catalyst composition described herein in a reactor system. The catalyst composition may be prepared outside of the reactor system and then loaded into it, or prepared inside the reactor system before being contacted with the hydrocarbon-containing feedstock.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

FIG. 1 shows the UMAP reduction of MBTR features of zeolite frameworks. Red circles are the zeolite clusters from which the zeolites investigated in this work have been derived.

FIG. 2 shows the self-diffusion coefficient and adsorption energy of ethane in zeolites at 350° C.

FIG. 3 shows the effect of different zeolite frameworks in ethane oxidative dehydrogenation at 350° C.

FIG. 4 shows the propylene formation rate as a function of time-on-stream during the reaction step (of two-step chemical looping process) measured in a lab-scale plug flow reactor on Cu-based MOR zeolite and Zn-based MOR catalysts.

FIG. 5 shows the energy barriers for the C—H bond activation as a function of metal species.

### DETAILED DESCRIPTION

Aspects of the present disclosure are best understood by reference to the description set forth herein. All the aspects described herein will be better appreciated and understood when considered in conjunction with the following descriptions. It should be understood, however, that the following descriptions, while indicating preferred aspects and numerous specific details thereof, are given by way of illustration only and should not be treated as limitations. Changes and modifications may be made within the scope herein without departing from the spirit and scope thereof, and the present disclosure herein includes all such modifications.

Catalytic Composition with MTW Framework Cluster Zeolite

A catalyst composition comprises a metal catalyst chemically interacted with a zeolite comprising at least one zeolite framework selected from the group of clusters consisting of MTW framework cluster, wherein the MTW framework cluster comprises zeolite frameworks having a Euclidean distance of 2.45 Ångström or less from the MTW framework.

For the purposes of the present disclosure, the term “chemically interacted” means that two or more molecules, atoms, or ions in the metal catalyst and the zeolite undergo a chemical interaction that leads to the formation of a bond. The bond can be, for example, a covalent bond, an ionic bond, a metallic bond, or a hydrogen bond. For the purposes of the present disclosure, the metal catalyst may be in the oxidized state or in any transition state. In one or more embodiments, the metal of the metal catalyst is in the oxidized form (i.e., one or more of its cationic forms).

A zeolite is a microporous, crystalline aluminosilicate material mainly consisting of silicon, aluminum, and oxygen, typically having the general formula Mⁿ⁺_{1/n} (AlO₂)⁻ (SiO₂)ₓ·yH₂O, where Mⁿ⁺_{1/n} is either a metal ion or H⁺, x is Si/Al molar ratio (or SiO₂/AlO₂ molar ratio) and is greater than 1, and y is the number of water molecules in the formula unit. In one or more embodiments, the zeolite is a cationic zeolite. In one or more embodiments, the zeolite according to the present invention is a protonic zeolite, i.e., M is H+.

The framework cluster is defined considering the structure similarity between different zeolite frameworks. The structures are featurized using Many-Body Tensor representation (MBTR) method as disclosed in Huo, H., & Rupp, M. (2022). Unified representation of molecules and crystals for machine learning. Machine Learning: Science and Technology, 3(4), 045017, incorporated herein by reference. The featurized structures are then dimensionally reduced using Uniform Manifold Approximation and Projection (UMAP) as disclosed in McInnes, L., Healy, J., & Melville, J. (2018). Umap: Uniform manifold approximation and projection for dimension reduction. arXiv preprint arXiv:1802.03426, incorporated herein by reference. Subsequently. the Euclidean distance is calculated between the reference framework and other zeolites, creating a ranked list of zeolites, ordered by their structural resemblance to the reference framework based on the value of the Euclidean distance. For the purposes of the present disclosure, a “framework cluster” is defined as a set of zeolite frameworks having a similarity to the reference framework based on a Euclidean distance of 2.45 Ångström or less from the reference framework.

The Euclidean distance is a measure of the straight-line distance between two points in Euclidean space. It's calculated using the Pythagorean theorem. For two points ((x1, y1)) and ((x2, y2)) in a two-dimensional plane, the Euclidean distance (d) is given by Equation (I):

$$d = \sqrt{{( {x}_{2} - {x}_{1} )}^{2} + {( {y}_{2} - {y}_{1} )}^{2}} \qquad ( I )$$

The inventors have particularly verified that, when the zeolite frameworks are selected from the MTW framework cluster, the combination of parameters related to the alkanes, such as the self-diffusion coefficient of ethane and adsorption energy of ethane are advantageous over other zeolite frameworks. Typically, such parameters correlate with higher catalytic activity towards olefins, making it a reliable metric for zeolite framework selection.

In one or more embodiments, the metal catalyst in the catalyst composition is selected from at least one transition metal, at least one alkaline earth metal, or any combination thereof. In a preferred embodiment, the metal catalyst in the catalyst composition comprises at least one transition metal selected from Groups 5 to 12 of the Periodic Table of Elements. Transition metals from these groups offer a wide range of catalytic properties due to their partially filled d-orbitals, which allow for various oxidation states and coordination geometries. The at least one transition metal in the catalyst composition may be selected from the group consisting of V, Cr, Mn, Fe, Co, Ni, Cu, Mo, Zn, W, and any combination thereof. In a specific embodiment, the transition metal in the catalyst composition is Cu.

In one or more embodiments, the metal catalyst in the catalyst composition comprises at least one alkaline-earth metal. The at least one alkaline earth metal in the catalyst composition may be selected from the group consisting of Be, Mg, Ca, Sr, Ba, or any combination thereof.

Zeolite frameworks contained in the MTW cluster may be selected from the group consisting of GON, SFN, SFH, VFI, AHT, SFE, SSY, MTT, OSI, TON, CFI, JBW, BIK, MON, CAS, APD, NSI, DFT, ATV, NAT, THO, EDI, NON, BCT, MEP, MTN and DOH. In one or more embodiments, the zeolite framework in the catalyst composition can be a GON zeolite framework. The GON framework belongs to the MTW framework cluster and exhibits an Euclidean distance of 0.77 Ångström from the MTW framework. This structural similarity allows for comparable catalytic performance while offering potential advantages in terms of stability or activity. The GON and other zeolite frameworks cited herein can be synthesized using various templating agents and synthesis conditions known in the art.

In one or more embodiments, the catalyst composition has a self-diffusion coefficient of ethane ranging from 5×10⁻⁹ m²/s to 10×10⁻⁸ m²/s, at 350° C., preferably from 1×10⁻⁸ m²/s to 6×10⁻⁸ m²/s, more preferably from 3×10⁻⁸ m²/s to 6×10⁻⁸ m²/s. The self-diffusion coefficient translates how the molecules move within the porous structure of the zeolite without an external concentration gradient, driven by thermal energy. Self-diffusion affects how reactants reach the active catalytic sites within the zeolite and governs how quickly products diffuse out of the zeolite. For the self-diffusion of ethane in the context of alkane conversion to olefins, a high diffusion self-diffusion coefficient of ethane molecules facilitates their conversion to ethylene and other valuable olefins. The self-diffusion coefficient may be measured by techniques known by a skilled person such as Pulsed Field Gradient Nuclear Magnetic Resonance (PFG-NMR), Quasi-Elastic Neutron Scattering (QENS), and Molecular Dynamics Simulations.

In one or more embodiments, the catalyst composition has adsorption energy of ethane in a range of from −10 kJ/mol to −30 kJ/mol at 350° C., preferably from −14 kJ/mol to −20 kJ/mol at 350° C. The adsorption energy describes the strength of the interaction between a molecule and the zeolite surface. For reactants, if adsorption energy is too low, reactants may not bind effectively, reducing catalytic efficiency. For products, if the adsorption energy is too high, desorption of products might be hindered, slowing down the reaction. The adsorption energy may be measured by techniques known by a skilled person such as calorimetry (DSC), inferred from adsorption isotherms or computational methods applying methods such as density functional theory and Widom insertion method as described in Widom, B. (1963). Some topics in the theory of fluids. The Journal of Chemical Physics, 39(11), 2808-2812, incorporated herein by reference.

In one or more embodiments, the catalyst composition has a micropore volume in a range of at least 0.03 cm³/g, for example from 0.03 cm³/g to 0.3 cm³/g determined from Ar adsorption isotherms measured at 87 K. Micropores are defined as the smallest pores within the catalyst, having diameters smaller than 2 nm.

In one or more embodiments, the catalyst composition has a surface area of about 100 cm²/g to about 400 cm²/g, determined from Ar adsorption isotherms measured at 87K.

Catalytic Composition with Alkaline-Earth Metal Catalyst

In another aspect, the present disclosure relates to a catalyst composition comprising at least one metal catalyst chemically interacted with a zeolite comprising at least one zeolite framework, wherein the at least one metal catalyst is selected from alkaline earth metal. In one or more embodiments, the at least one alkaline earth metal in the catalyst composition is selected from the group consisting of Be, Mg, Ca, Sr and Ba.

The inventors have particularly verified that, when the metal species chemically interacted with the zeolite are selected from the alkaline-earth metal group, the activation energy for the C—H bond activation is lower when compared to usual transition metal catalysts. Usual high-throughput screening (HTS) has the C—H bond activation energy as one of the key descriptors for studying different metal-zeolites combinations for alkanes to olefins conversions. Typically, lower activation energy correlates with higher catalytic activity, making it a reliable metric for metal catalyst selection.

The zeolite framework of the catalyst composition comprising at least one alkaline-earth metal catalyst is not particularly limited and may be selected from a variety of zeolite frameworks. Suitable zeolite frameworks include, but are not limited to MFI (such as ZSM-5), MEL (such as ZSM-11), MOR (such as mordenite), BEA (such as Beta), FER (such as ferrierite), LTL (such as Linde type), FAU (such as Y-zeolite), TON (such as theta-1), MTW (such as ZSM-12) and GON (such as GUS-1). In one or more embodiments, the zeolite framework is selected from MTW, GON, or MOR.

In one or more embodiments, the catalyst composition has a micropore volume in a range of at least 0.03 cm³/g, for example from 0.03 cm³/g to 0.3 cm³/g determined from Ar adsorption isotherms measured at 87 K. Micropores defined are the smallest pores within the catalyst, having diameters smaller than 2 nm.

In one or more embodiments, the catalyst composition has a surface area of about 100 cm²/g to about 400 cm²/g, determined from Ar adsorption isotherms measured at 87K.

### Process for Preparing the Catalyst Composition

The catalyst composition according to the present disclosure can be prepared through various methods, including ion exchange, impregnation, or co-precipitation with the zeolite support. In one or more embodiments, the catalyst composition according to the present disclosure is prepared by the wet impregnation method. In one ore more embodiments, the catalyst composition according to the present disclosure is prepared by solid-state ion-exchange procedure. The catalyst composition may be prepared either inside or outside of a reactor system.

In one particular aspect, the present disclosure relates to a process for preparing a catalyst composition comprises adding, to a zeolite comprising at least one zeolite framework selected from the group of clusters consisting of MTW framework cluster, a metal catalyst precursor to form a catalyst precursor mixture; and heating the catalyst precursor mixture to a temperature of from 390° C. to 750° C. to form the catalyst composition, wherein the MTW framework cluster comprises zeolite frameworks having a Euclidean distance of 2.45 Ångström or less from the MTW framework. In one or more embodiments, the heating may occur at a temperature of about 350° C. to about 450° C.

All the above descriptions and all embodiments discussed in the above aspects relating to the catalyst composition described herein, such as MTW framework cluster, metal catalyst, and catalyst properties apply to this aspect of the invention relating to the process for preparing the catalyst composition.

In one or more embodiments, the metal catalyst precursor is selected from at least one transition metal precursor, at least one alkaline earth metal precursor, or any combination thereof.

It is envisioned that the metal catalyst precursor may comprise the at least one metal to be incorporated into the catalyst in the form of a metal compound such as transition metal compounds, alkaline-earth metal compounds or mixtures thereof, or alloys formed between one or more metals according to the present disclosure. In one or more embodiments, the at least one metal is incorporated as the oxidized form (i.e., one or more of its cationic forms), or any transition state.

Preferably, the metal catalyst precursors may be in the form of oxides, hydroxides or salts such as carbonates, nitrates, acetates and metal salts of an organic acid, or mixtures thereof, with the metal being in the oxidized form or any transition state. In particular embodiments, transition metal catalyst precursors according to the present disclosure may be selected from transition metal oxides, transition metal hydroxides or transition metal salts such as transition metal carbonates, transition metal nitrates, transition metal acetates and transition metal salts of an organic acid, or mixtures thereof, with the metal being in the oxidized form or any transition state. In particular embodiments, alkaline-earth metal catalyst precursors according to the present disclosure may be selected from alkaline-earth metal oxides, alkaline-earth metal hydroxides or alkaline-earth metal salts such as alkaline-earth metal carbonates, alkaline-earth metal nitrates, alkaline-earth metal acetates and alkaline-earth metal salts of an organic acid, or mixtures thereof, with the metal being in the oxidized form or any transition state.

In a preferred embodiment, the metal catalyst precursor comprises at least one transition metal selected from Groups 5 to 12 of the Periodic Table of Elements. The at least one transition metal precursor may be selected from the group consisting of V precursor, Cr precursor, Mn precursor, Fe precursor, Co precursor, Ni precursor, Cu precursor, Mo precursor, Zn precursor, W precursor, and any combination thereof. In a specific embodiment, the transition metal precursor in the catalyst is Cu precursor.

In one or more embodiments, the metal catalyst precursor in the catalyst composition comprises at least one alkaline-earth metal precursor. The at least one alkaline earth metal precursor in the catalyst composition may be selected from the group consisting of Be precursor, Mg precursor, Ca precursor, Sr precursor, Ba precursor, or any combination thereof.

In another particular aspect, the present disclosure relates to a process for preparing a catalyst composition comprising adding, to a zeolite comprising at least one zeolite framework, an alkaline earth metal catalyst precursor to form a catalyst precursor mixture; and heating the catalyst precursor mixture to a temperature of from 390° C. to 750° C. to form the catalyst composition. In one or more embodiments, the heating may occur at a temperature of about 350° C. to about 450° C.

All the above descriptions and all embodiments discussed in the above aspects relating to the catalyst composition described herein, such as metal catalyst, zeolite framework, and catalyst properties apply to this aspect of the invention relating to the process for preparing the catalyst composition.

In one or more embodiments, the alkaline earth metal catalyst precursor in the catalyst composition comprises at least one alkaline-earth metal precursor. The at least one alkaline earth metal precursor in the catalyst composition may be selected from the group consisting of Be precursor, Mg precursor, Ca precursor, Sr precursor, Ba precursor, or any combination thereof.

Preferably, the alkaline earth metal catalyst precursors may be in the form of oxides, hydroxides or salts such as carbonates, nitrates, acetates and metal salts of an organic acid, or mixtures thereof, with the metal being in the oxidized form or any transition state. In particular embodiments, alkaline-earth metal catalyst precursors according to the present disclosure may be selected from alkaline-earth metal oxides, alkaline-earth metal hydroxides or alkaline-earth metal salts such as alkaline-earth metal carbonates, alkaline-earth metal nitrates, alkaline-earth metal acetates and alkaline-earth metal salts of an organic acid, or mixtures thereof, with the metal being in the oxidized form or any transition state.

### Process for Catalytic Oxidative Dehydrogenation of Hydrocarbons

The catalyst composition of the present disclosure finds primary application in the catalytic oxidative dehydrogenation of hydrocarbons, particularly in the conversion of light alkanes to light olefins. This process involves contacting a hydrocarbon-containing feedstock with the catalyst composition in a reactor system. The feedstock typically comprises refinery-range hydrocarbons, with a focus on light alkanes such as ethane, propane, n-butane, n-pentane, n-hexane, n-heptane, and n-octane, as well as their isomers. The versatility of the catalyst composition allows for the efficient conversion of C2-C8 alkanes, linear or branched, either individually or in combinations, to their corresponding olefinic compounds.

In one aspect, the present disclosure relates to a process for catalytic oxidative dehydrogenation of hydrocarbons according to the present disclosure comprises contacting, in a reactor system, a hydrocarbon-containing feedstock with any of the catalyst compositions described herein. This process utilizes the catalyst compositions described herein to convert light alkanes into light olefins in a low-emission and cost-effective manner. The catalyst composition, comprising a metal catalyst chemically interacted with a zeolite framework, provides high activity for the oxidative dehydrogenation reaction.

All the above descriptions and all embodiments discussed in the above aspects relating to the catalyst compositions described herein and the process for preparing such catalysts, such as metal catalyst, zeolite framework, framework cluster, catalyst properties, catalyst synthesis steps, and operation conditions apply to this aspect of the invention relating to the process for catalytic oxidative dehydrogenation of hydrocarbons.

In one or more embodiments, the catalyst composition for the catalytic oxidative dehydrogenation process may be prepared outside of the reactor system and then loaded into the reactor system (ex-situ preparation). Once prepared, the catalyst is loaded into the reactor system. This approach enables the use of complex preparation techniques that may not be feasible within the reactor system itself.

In one or more embodiments, the catalyst composition for the catalytic oxidative dehydrogenation process may be prepared inside the reactor system before being contacted with the hydrocarbon-containing feedstock. This in-situ preparation method offers several advantages, including the ability to generate fresh catalyst surfaces immediately before the reaction. The catalyst precursors can be loaded into the reactor system, and the final catalyst composition is formed under controlled conditions within the reactor. This approach can be particularly beneficial for catalysts that are sensitive to air or moisture exposure. In-situ preparation also allows for the possibility of dynamic catalyst modification during the reaction process, potentially leading to improved catalytic performance and longer catalyst lifetime.

In one or more embodiments, the process for catalytic oxidative dehydrogenation of hydrocarbons is exothermic. The conversion of light alkanes to olefins through oxidative dehydrogenation releases heat due to the formation of chemical bonds in the products that are stronger than those in the reactants.

The process for catalytic oxidative dehydrogenation of hydrocarbons is conducted at a temperature of about 750° C. or less, preferably from about 20° C. to about 750° C., more preferably from about 20° C. to about 500° C., more preferably from about 20° C. to about 450° C., even more preferably from about 20° C. to about 400° C., even more preferably from about 20° C. to about 350° C.

In one or more embodiments, the process is carried out at pressures of up to 5 atm, with a preferred range of 1 atm to 3 atm. The process may be a continuous process, a semi-continuous process, or a batch process.

In one or more embodiments, the olefinic compounds produced by the catalytic oxidative dehydrogenation of the present disclosure comprise light olefins, α-olefins, terminal dienes, or any combination thereof. In one or more embodiments, the olefinic compounds are selected from the group consisting of ethene, propene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, and 1,5-hexadiene.

In one or more embodiments, the hydrocarbon-containing feedstock in the process comprises refinery range hydrocarbon, which includes at least one light alkane. The light alkane may be a C2-C8 alkane, linear or branched, or any combination thereof, such as ethane, propane, n-butane, n-pentane, n-hexane, n-heptane, n-octane, its isomers, or combinations thereof.

The process can be conducted in the presence of an oxygen source, which may comprise a purified 02 stream, an air stream, or any combination thereof. In one or more embodiments, one or more diluents, such as nitrogen, argon, or helium, can be included. The reactor system used in the process may comprise a single reactor or multiple reactors connected in a continuous loop for catalyst circulation. In the case of a single reactor system, the catalyst composition can be contacted sequentially with the hydrocarbon-containing feedstock and then with the oxygen source.

The catalyst composition and associated process offer significant advantages over traditional methods of light alkane conversion. The improved catalytic efficiency results in higher yields of desired olefin products while minimizing the formation of unwanted byproducts. The ability to operate at lower temperatures compared to conventional cracking processes leads to substantial energy savings and reduced greenhouse gas emissions. Furthermore, the enhanced stability and longer lifetime of the catalyst composition contribute to reduced operational costs and improved process economics.

The present disclosure represents a significant advancement in the field of heterogeneous catalysis, particularly in the context of light alkane conversion. By combining the unique properties of zeolite frameworks and/or selected metal catalysts, the invention provides a powerful tool for addressing the growing demand for light olefins while simultaneously improving the sustainability and efficiency of industrial processes.

The embodiments of the present disclosure disclosed herein are intended to be illustrative and not limiting. Other embodiments are possible and modifications may be made to the embodiments without departing from the spirit and scope of the invention. As such, these embodiments are only illustrative of the inventive concepts contained herein.

### EXAMPLES

The following examples are provided to illustrate embodiments of the present disclosure. The Examples are not intended to limit the scope of one or more embodiments of the present disclosure, and they should not be so interpreted.

### Example 1—Zeolite Frameworks Clustering and Evaluation

### 1.1. Extracting Similar Structures

IZA database available at https://www.iza-structure.org/databases/, which contains 255 experimental zeolite structures was used. These structures were featurized using the Many-Body Tensor Representation (MBTR) method as disclosed in Huo, H., & Rupp, M. (2022). Unified representation of molecules and crystals for machine learning. Machine Learning: Science and Technology, 3(4), 045017. The MBTR method was implemented in Describe software described in Himanen, L., Jager, M. O., Morooka, E. V., Canova, F. F., Ranawat, Y. S., Gao, D. Z., . . . & Foster, A. S. (2020). DScribe: Library of descriptors for machine learning in materials science. Computer Physics Communications, 247, 106949, incorporated herein by reference. The featurized structures are then dimensionally reduced to cluster zeolites in feature space using Uniform Manifold Approximation and Projection (UMAP) as disclosed in McInnes, L., Healy, J., & Melville, J. (2018). Umap: Uniform manifold approximation and projection for dimension reduction. arXiv preprint arXiv:1802.03426.

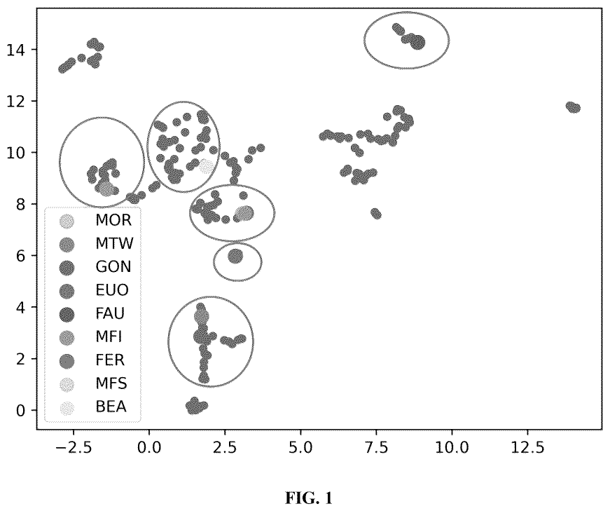

Clusters of zeolites were identified through UMAP feature reduction of MBTR features as shown in FIG. 1. For the MBTR descriptor used in this study, the following parameters were specified:

Species: The descriptor was configured to consider Silicon (Si) and Oxygen (O) atoms, which are the primary constituents of zeolite frameworks.

Geometry Function: The cosine function was employed to calculate the geometric features, which is particularly useful for capturing angular relationships between atoms.

Grid: The minimum value of grid for the geometric features was set to start at −1, ensuring that the full range of the cosine function is utilized. The maximum value of the grid extends to 1, which is the upper limit for the cosine function. A total of 100 points were used to discretize the grid, providing a detailed representation of the geometric features. A value of 0.1 was used for the Gaussian smearing parameter, sigma, which helps in smoothing the distribution and reducing noise.

Weighting Function: An exponential weighting function was applied with a scale of 0.5 and a threshold of 1e-3. This emphasizes the importance of closer atomic pairs over distant ones, which is critical for capturing the local chemical environment.

Periodicity: The descriptor was set to periodic, acknowledging the repeating nature of the zeolite crystal structure, which is essential for accurately representing the material.

Normalization: L2 normalization was used to ensure that the length of the feature vector is normalized, which aids in comparing features across different structures.

The simulations identified zeolite structures within the IZA database that are structurally analogous to selected reference zeolite frameworks such as MOR, FAU, MFI, BEA, EUO and MTW. To achieve this, a dimensionality reduction technique known as UMAP was applied to the MBTR-derived features of the zeolites. Subsequently, the Euclidean distance—a measure of similarity—was calculated between the reference zeolite frameworks and each of the other zeolites. This calculation facilitated the creation of a ranked list of zeolites, ordered by their structural resemblance to the selected frameworks. The zeolites that exhibited the closest structural affinity to the selected frameworks were deemed the most similar.

### 1.2. Self-Diffusion Coefficient

Molecular dynamics simulations were conducted to determine the self-diffusivity of ethane in zeolites using the LAMMPS software, as described in Plimpton, S, J. Comput. Phys. 117 (1995),1, incorporated herein by reference. Initial configurations with 20 ethane molecules were generated using the Cassandra Software, described in Shah, J. K., Marin-Rimoldi, E., Mullen, R. G., Keene, B. P., Khan, S., Paluch, A. S., . . . & Maginn, E. J. (2017). Cassandra: An open source Monte Carlo package for molecular simulation, incorporated herein by reference. The TraPPE forcefield for ethane, as described in Chen, B., & Siepmann, J. I. (1999). Transferable potentials for phase equilibria. 3. Explicit-hydrogen description of normal alkanes. The Journal of Physical Chemistry B, 103(25), 5370-5379, incorporated herein by reference, and the TraPPE-zeo forcefield, as described in Bai, P, Tsapatsis, M, and Siepman, J, L, J. Phys. Chem. C., 117 (2013), incorporated herein by reference, for zeolite frameworks were employed. Simulations were performed at 350° C., with the framework held rigid and only ethane molecules allowed to move in the NVT (constant number of molecules, constant volume, and constant temperature) ensemble. The Nose-Hoover Thermostat, as described in Hoover, W. G. (1985). Canonical dynamics: Equilibrium phase-space distributions. Physical review A, 31(3), 1695, incorporated herein by reference, was used to maintain the temperature of the system. A timestep of 1 femtosecond was used, and simulation was run for a total of 20 nanoseconds from which the trajectories were recorded every 20 picoseconds for calculation of self-diffusion coefficient. The Einstein self-diffusion coefficient was calculated using equation (II):

$$D = {lim}_{t \to ∞} \frac{1}{2 dt} 〈 {\vert r ( t ) - r ( 0 ) \vert}^{2} 〉 \qquad ( II )$$

Where r(0) and r(t) are the positions of molecules at time 0 and t, respectively, and d is the dimensionality of the system. The mean square displacement was computed in each direction to determine the system's dimensionality. Final self-diffusion coefficient was computed by averaging self-diffusion coefficient results from four independent simulations.

### 1.3. Adsorption Energy

Adsorption energy was determined using the Widom insertion method as described in Widom, B, J. Chem. Phys., 39(1963), 2808 with the RASPA software as described in Dubbeldam, D, Calero, S, Ellis, D, E, and Snurr, R, Q, Mol. Sim., 42 (2016), 81, incorporated herein by reference, at 350° C. The same forcefields as mentioned above were used. Simulations ran for 10,000 cycles, with one cycle defined as the number of molecules multiplied by the Monte Carlo step. A Widom probability of 1 were used. Adsorption energy is computed using equation (III):

$$Δ H = {U}_{gh} - {U}_{h} - {U}_{G} - RT \qquad ( III )$$

Where ΔH is adsorption energy, U_{gh} is the average energy of the guest molecule within the host framework, Uₕ is the average energy of the host framework, U_{g} is the energy of the single guest molecule in the gas phase and RT is the enthalpy per particle of the ideal bulk phase. Since the framework and molecules bot are rigid, Uₕ=0 and U_{g}=0.

### 1.3.1 Self-Diffusion Coefficient and Adsorption Energy Results

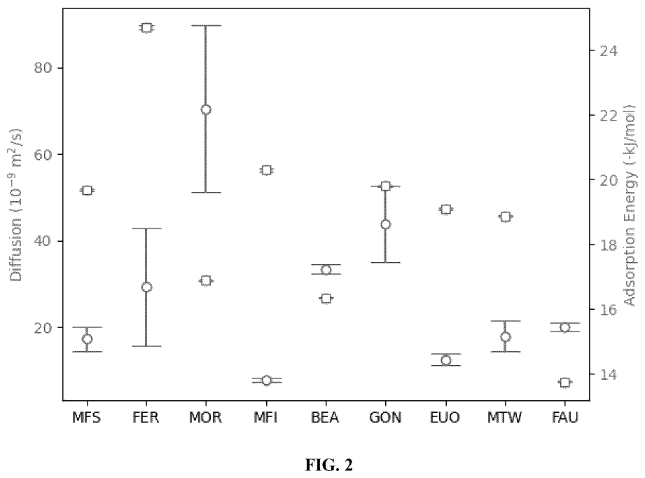

In the study, Mordenite (MOR) demonstrated the greatest self-diffusivity of ethane, as depicted in FIG. 2. Conversely, Ferrierite (FER) showed the highest adsorption energy for ethane.

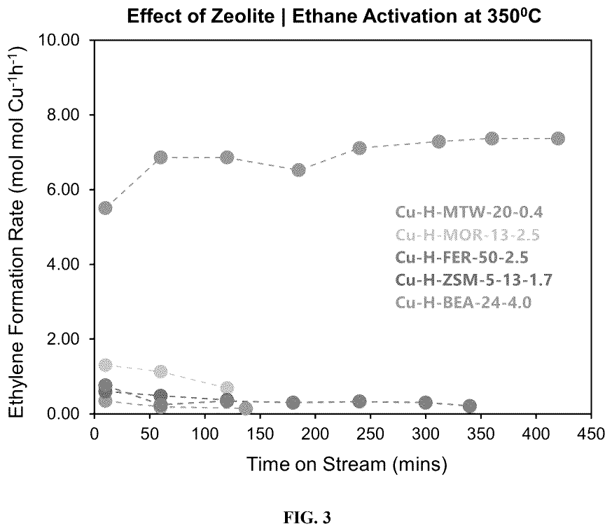

For the assessment and comparison of the self-diffusion coefficient and adsorption energy results, data from real bench scale oxidative dehydrogenation of ethane to ethylene was used as a reference. These results are shown in FIG. 3.

For these results, catalysts were synthesized via a solid-state ion-exchange procedure. In case of Cu-based MOR zeolite version of the catalyst (hereinafter referred to as Cu—H-MOR), Cu was incorporated on commercial microporous MOR support (CBV21A, NH4-MOR, Zeolyst) via solid-state ion-exchange, wherein 0.08 g of copper nitrate precursor was physically mixed with 5.92 g of H-MOR microporous zeolite and grounded in a mortar and pestle for about 15 mins or till the solid mixture turns light green or seems homogenous. This solid mixture was then heated to 873K (0.167 K s-1) under the flow of air in a horizontal tube furnace and held for 6 h before cooling to room temperature. The same synthesis procedure was proposed for other zeolites (MTW, FER, ZSM, BEA).

For catalyst activity tests, ethane was fed onto a bed of transition metal incorporated high-silica microporous mixed metal oxide (0.2 g) contained in a lab-scale plug flow reactor. The catalyst bed was maintained at a temperature of 350° C. Ethane feed concentration was about 35% in nitrogen and total feed rate was about 200 ml/min. FIG. 3 illustrates the results comparing the ethylene formation rate as a function of time-on-stream at 350° C. on various Cu-based zeolite catalysts.

Notably, even with FER's superior adsorption energy, MOR outperformed in terms (FIG. 3) of ethylene production, underscoring the pivotal role of ethane's diffusive properties in the activation of alkanes. Meanwhile, Merlinoite (MTW), which structurally differs from MOR, BEA, and MFI, surprisingly registered the highest rate of ethylene conversion (FIG. 3). Additionally, Gobbinsite (GON), which shares similarities with MTW in the UMAP feature space, exhibited both higher adsorption energy and a higher self-diffusion coefficient for ethane compared to MTW. This indicates that GON may have an even greater capacity for ethylene conversion than MTW and the other zeolites studied.

### Example 2—Alkaline-Earth Metal Catalyst Evaluation

### 2.1. Computational High-Throughput Screening

Several calculations were used to elucidate the reactivity on metal-exchanged MOR. In converting alkanes to olefins, the C—H bond activation energy is essential for studying the reactivity of different metal centers on zeolites. The bond activation energy refers to the minimum amount of energy required to initiate a chemical reaction by breaking the bonds of the reactants. This energy is necessary to reach the transition state, where reactants are transformed into products. Here lower activation energy typically correlates with higher catalytic activity, making it a reliable metric for initial screening. Utilizing C—H bond activation energy as a metric for studying metal centers on zeolites in the alkane-to-olefins process is a valid and practical approach. This approach was used to screen several metal centers on the MOR zeolite. To determine the activation energy for C—H bond activation energy on a reactive metal center in a zeolite framework, calculations were performed using the M3GNET universal potential. All calculation herein were completed using the M3GNET universal potential as described in Chen, C., Ong, S. P. A universal graph deep learning interatomic potential for the periodic table. Nature Computational Science, 2023, 2, 718-728. with the Pymatgen software as described in Ong, S. P., Cholia, S., Jain, A., Brafman, M., Gunter, D., Ceder, G., & Persson, K. A. (2015). Python Materials Genomics (pymatgen): A robust, open-source python library for materials analysis. Computational Materials Science, 68, 314-3. The study began by selecting a representative zeolite structure containing the reactive metal center (e.g., alkali earth metal) to serve as the catalytic site. The zeolite framework was modeled as a periodic system and the reactant molecule, such as a hydrocarbon, was positioned with its C—H bond oriented toward the metal center to facilitate interaction. Geometry optimizations were conducted as a preliminary step to refine the system configuration.

The M3GNET universal potential was employed to describe the complex interactions within the system, including those involving the zeolite framework, the reactive metal, and the hydrocarbon molecule. Transition state calculations for the C—H bond activation step was initiated by generating an initial transition state guess. This guess involved perturbing the geometry of the hydrocarbon to elongate the target C—H bond, guided by chemical intuition. The transition state geometry was subsequently optimized using M3GNET to locate the saddle point on the potential energy surface corresponding to C—H bond activation. The activation energy was calculated as the difference between the energy of the system at the transition state and the reactant state.

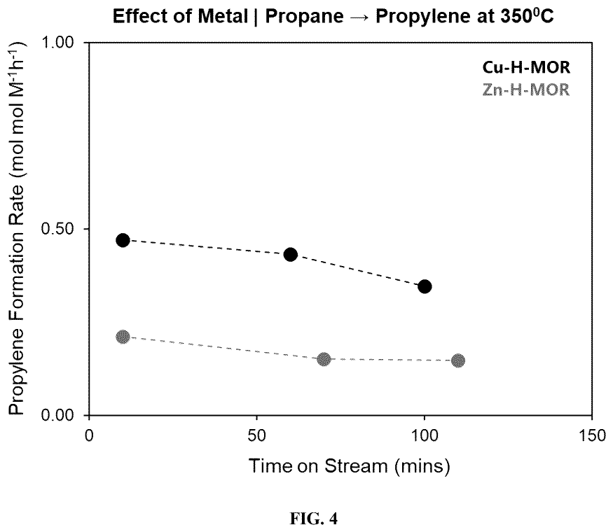

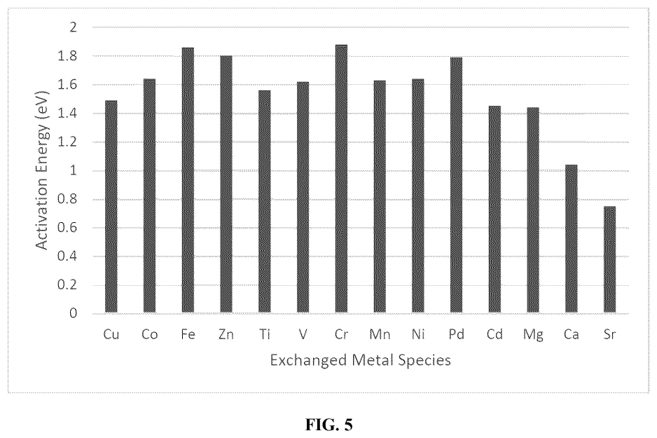

The calculations in FIG. 5 show that alkaline earth metals (such as Mg, Ca, and Sr) outperform other metal species. The method's validity is compared to experimental results shown in FIG. 4, which show Cu-exchanged MOR being superior to Zn-exchanged MOR. The calculations show the activation energy of Cu-exchanged MOR to be 1.5 eV and Zn-exchanged MOR as 1.8 eV, which indicates that both are aligned in that Cu-exchanged MOR is superior. Therefore, the alkaline earth metal exchanged zeolites are predicted to be superior to Cu-exchanged MOR.

### 2.2 Experimental Catalyst Synthesis:

For the Cu-based MOR zeolite version of the catalyst (hereinafter referred to as Cu—H-MOR), Cu was incorporated on commercial microporous MOR support (CBV21A, NH4-MOR, Zeolyst) via solid-state ion-exchange, wherein 0.08 g of copper nitrate precursor was physically mixed with 5.92 g of H-MOR microporous zeolite and grounded in a mortar and pestle for about 15 mins or till the solid mixture turns light green or seems homogenous. This solid mixture was then heated to 873K (0.167 K s-1) under the flow of air in a horizontal tube furnace and held for 6 h before cooling to room temperature.

For the Zn-based MOR zeolite version of the catalyst (hereinafter referred to as Zn—H-MOR), Zn was supported on the commercial MOR support (CBV21A, NH4-MOR, Zeolyst) using the same solid-state ion-exchange procedure.

### Experimental Catalyst Characterization:

Surface area and micropore volumes of the catalysts were determined from Ar adsorption isotherms measured at 87 K on a Micromeritics ASAP 2020 Surface Area and Porosity Analyzer. Typically, 0.03-0.05 g of pelleted and sieved sample (nominal diameter between 180-250 m) were degassed by heating to 120° C. (10° C./min) under vacuum (<5 mHg) for 2 h, and then further heating to 350° C. (10° C./min) under vacuum (<5 mHg) and holding for 9 h. Volumetric gas adsorption within micropores (cm3 g-1 at STP) was estimated from analysis of semi-log derivative plots of the adsorption isotherm (∂(Vads)/∂(ln(P/PO)) vs. ln(P/PO)) to identify the micropore filling transition (first maximum) and then the end of micropore filling (subsequent minimum). Micropore volumes (cm3 g-1) were obtained by converting standard gas adsorption volumes (cm3 gcat-1 at STP) to liquid volumes using a density conversion factor assuming the liquid density of Ar at −186° C. Surface area and micropore volumes of the catalysts are shown in Table 1 and are in line with what is expected for conventional microporous zeolite supports.

TABLE 1
Surface area and micropore volume measured on varying
zeolite and metal-based catalysts (Catalyst materials
are labelled as M-H-Zeolite-Silica to Alumina-M wt %)

| Sample | Surface Area (cm² g⁻¹) | Micropore Volume (cm³ g⁻¹) |
|---|---|---|
| Cu-H-MTW | 308 | 0.17 |
| Cu-H-MOR | 368 | 0.16 |
| Cu-H-FER | 318 | 0.18 |
| Cu-H-ZSM | 303 | 0.11 |
| Cu-H-BEA | 488 | 0.16 |

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. It is the express intention of the applicant not to invoke means-plus-function for any limitations of any of the claims herein, except for those in which the claim expressly uses the words ‘means for’ together with an associated function.

1. A catalyst composition comprising a metal catalyst chemically interacted with a zeolite comprising at least one zeolite framework selected from the group of clusters consisting of MTW framework cluster,
wherein the MTW framework cluster comprise zeolite frameworks having a Euclidean distance of 2.45 Ångström or less from the MTW framework.

2. The catalyst composition of claim 1, wherein the metal catalyst is selected from at least one transition metal, at least one alkaline earth metal, or any combination thereof.

3. The catalyst composition of claim 1, wherein the zeolite framework is a GON zeolite framework.

4. The catalyst composition of claim 1, having a self-diffusion coefficient of ethane in a range of from 5×10⁻⁹ m²/s to 10×10⁻⁸ m²/s at 350° C.

5. The catalyst composition of claim 1, having an adsorption energy of ethane in a range of from −10 kJ/mol to −30 kJ/mol at 350° C.

6. (canceled)

7. (canceled)

8. The catalyst composition of claim 1, wherein the metal catalyst comprises at least one transition metal selected from Groups 5 to 12 of the Periodic Table of Elements.

9. (canceled)

10. (canceled)

11. A catalyst composition comprising at least one metal catalyst chemically interacted with a zeolite comprising at least one zeolite framework, wherein the at least one metal catalyst is selected from alkaline earth metal.

12. (canceled)

13. (canceled)

14. (canceled)

15. A process for preparing a catalyst composition of claim 1, the process comprising:
adding, to a zeolite comprising at least one zeolite framework selected from the group of clusters consisting of MTW framework cluster, a metal catalyst precursor to form a catalyst precursor mixture; and
heating the catalyst precursor mixture to a temperature of from 390° C. to 750° C. to form the catalyst composition
wherein the MTW framework cluster comprise zeolite frameworks having a Euclidean distance of 2.45 Ångström or less from the MTW framework.

16. A process for preparing a catalyst composition of claim 11, the process comprising:
adding, to a zeolite comprising at least one zeolite framework, at least one alkaline earth metal catalyst precursor to form a catalyst precursor mixture; and
heating the catalyst precursor mixture to a temperature of from 390° C. to 750° C. to form the catalyst composition.

17. A process for catalytic oxidative dehydrogenation of hydrocarbons, the process comprising:
contacting, in a reactor system, a hydrocarbon-containing feedstock with the catalyst composition of claim 1 to produce one or more olefinic compounds.

18. (canceled)

19. (canceled)

20. (canceled)

21. (canceled)

22. (canceled)

23. The process of claim 17, wherein the process is a continuous process, a semi-continuous process, or a batch process.

24. The process of claim 17, wherein the olefinic compounds comprise light olefins, α-olefins, terminal dienes, or any combination thereof.

25. (canceled)

26. (canceled)

27. (canceled)

28. (canceled)

29. (canceled)

30. The process of claim 17, wherein the contacting is in the presence of:
an oxygen source, wherein the oxygen source comprises a purified O₂ stream, an air stream, or any combination thereof, and
optionally, a diluent selected from the group consisting of nitrogen, argon, and helium.

31. The process of claim 17, wherein the reactor system comprises a single reactor or at least a first reactor and a second reactor connected in a continuous loop for catalyst circulation.

32. The process of claim 17, wherein the reactor system comprises a single reactor, and the heterogeneous catalyst composition is contacted sequentially, first with the hydrocarbon-containing feedstock, then with the oxygen source.
